(19) United States
(12) Patent Application Publication
(10) Pub. No.: US 2007/0224637 A1
(43) Pub. Date: Sep. 27, 2007

(54) Oxidative protection of lipid layer biosensors

(76) Inventors: Joseph C. McAuliffe, Sunnyvale, CA (US); Mark S. Gebert, Pacifica, CA (US); Luis G. Cascao-Pereira, San Francisco, CA (US)

Correspondence Address: HOWREY LLP, C/O IP DOCKETING DEPARTMENT, 2941 FAIRVIEW PARK DRIVE, SUITE 200, FALLS CHURCH, VA, 22042-2924, US

(21) Appl. No.: 11/388,763

(22) Filed: Mar. 24, 2006

(51) Int. Cl. G01N 33/53; C12M 1/34

(52) U.S. Cl. 435/7.1; 435/287.2; 427/2.11

(57) ABSTRACT

The present invention provides methods and composition related to lipid layers with increased stability to oxidative degradation. In one embodiment, the invention provides lipid layers comprising antioxidant inclusion molecules that permit biosensors with increased performance and functional lifetime due to increased oxidative protection. In addition to these lipid-layer compositions, the present invention provides methods for further protection of lipid layers from oxidation. These methods include application of solutions and coatings comprising barrier compounds (e.g., trehalose), storage under anaerobic conditions (e.g., with inert gases), storage with oxidant scavengers (e.g., dessicants and catalysts), and packaging with barrier materials (e.g., mylar, polyethylene) that prevent oxidant exposure.

### FIELD OF THE INVENTION

The present invention relates to methods and compositions comprising lipid layers with increased stability to oxidative degradation.

### BACKGROUND OF THE INVENTION

Lipid layers, which are best known as the materials of cellular membranes, have been engineered and incorporated into numerous man-made products. Lipid layers find utility in numerous applications typically as a material or component that provides a boundary between two aqueous solutions. In particular, lipid layers which can be selectively permeable (e.g., bilayer, membranes) find use in sensing devices where movement of molecules across a barrier creates a signal. Biosensors are one form of sensing device that is used to detect biological molecules, typically a receptor ligand in a solution.

One form of biosensor that incorporates lipid layers are ion channel switch (ICS) biosensors. ICS biosensors have been described in U.S. Pat. Nos. 5,874,316; 5,234,566; 5,443,955; 5,741,409, 5,401,378; 5,637,201; 5,753,093; 5,783,054; 6,316,273; 6,451,196; 6,573,109; 5,741,712; U.S. patent application Ser. No. 11/024,571, filed Dec. 28, 2004; and published PCT application WO 98/55853; each of which is hereby incorporated by reference herein in its entirety. Briefly, ICS biosensors have a lipid bilayer membrane tethered (i.e., attached through a linker or spacer) to a solid support that is typically gold coated. The bilayer has inclusions of ion channel molecules (e.g., gramicidin), and biological receptor molecules (e.g., antibodies) are attached to the surface of the lipid layer distal to the support. The lipid bilayer is stabilized by tethering the first layer lipids to a gold electrode solid support via thiol chemistry and an intervening hydrophilic linker. The space between the first layer and the solid support creates a reservoir for ions at the electrode surface (Knoll, et al., J Biotechnol 74, 137-58 (2000); and Krishna, et al., Langmuir 17, 4858-4866 (2001)).

ICS biosensor detection of analyte is achieved by attaching antibody fragments to the mobile second (i.e., outer) layer gramicidin channels. Complementary antibody fragments are also attached to stationary membrane-spanning lipids that are tethered to the gold electrode. When an analyte is captured between the two antibody fragments, the mobile gramicidin channels of the outer layer are thereby anchored to the stationary lipids preventing the formation of conductive dimeric channels. The reduction in number of total available gramicidin dimers results in a rapid decrease in current across the membrane. This switching mechanism provides the means for the translation of a single biological event (e.g., the binding of analyte to a pair of analyte-recognizing antibody fragments) into a significantly amplified electrical signal (e.g., a change of flux of 10⁶ ions/sec per channel), a degree of amplification that can be used in creating a sensitive assay platform. This detection mechanism gives reliable quantitative results for the detection of a variety of analytes. For more information on the use of ICS biosensors see, e.g., Cornell, B. et al., Nature 387, 580-3 (1997); Came, S., ‘The Ambri Biosensor—A comprehensive Guide’ (2002), AMBRI Pty. Ltd, Australia; Cornell, et al., Optical Biosensors Present and Future (eds. F., L. & C., R. T.) 457 (Elsevier, Amsterdam, 2002).

In addition to lipid bilayer based sensors, lipid layers find use in devices employing monolayers. For example, self-assembled monolayers (SAMs) with tethered proteins or enzymes have been used as amperometric or coulometric sensing devices (see, e.g., Porter et al., “An electro-active system of immuno-assay (EASI assay) utilizing self assembled monolayer modified electrodes,” Biosens Bioelectron., 16(9-12):875-85 (2001); Campuzano et al., “An integrated electrochemical fructose biosensor based on tetrathiafulvalene-modified self-assembled monolayers on gold electrodes,” Anal Bioanal Chem., 377(4):600-7 (2003); and U.S. Pat. No. 6,241,863) or in surface plasmon resonance (SPR) sensor applications (see, e.g., Sigal et al., “A self-assembled monolayer for the binding and study of histidine-tagged proteins by surface plasmon resonance,” Anal Chem., 68(3):490-7 (1996)). Additional applications of SAMs in biosensor or electronic device applications are described in e.g., Chaki and Vijayamohanan, “Self-assembled monolayers as a tunable platform for biosensor applications,” Biosens Bioelectron. 17(1-2): 1-12 (2002); Osaka, “Creation of highly functional thin films using electrochemical nanotechnology,” Chem. Rec. 4(6):346-62 (2004); Schaeferling et al., “Application of self-assembly techniques in the design of biocompatible protein microarray surfaces,” Electrophoresis, 23(18):3097-105 (2002); and Vuillaume, “Nanometer-scale organic thin film transistors from self-assembled monolayers,” J Nanosci Nanotechnol. 2(3-4):267-79 (2002). PCT publication WO2005089415 describes methods for stabilizing SAMs on solid surfaces by adding a small amount of amphiphilic molecules, such as DMF and DMSO, into aqueous solutions as preserving media.

Lipid molecules, in particular those that include at least one unsaturated carbon-carbon bond, are susceptible to reacting with oxidants, such as molecular oxygen. Generally, lipid oxidation occurs when electrons are removed from the lipid molecule accompanied the loss of hydrogen or the addition of oxygen to the lipid molecule. A common mechanism for oxidation of lipids is “autoxidation.” Autoxidation involves an autocatalytic free-radical induced chain reaction of the lipid molecule (or other compound) with molecular oxygen. Autoxidation can be further catalyzed in the presence of certain metals, especially those with two or more valency states (e.g., cobalt, copper, iron, nickel), hydrogen ion (i.e., H⁺), or hydroxyl ion (i.e., OH⁻), which affect pH related redox potential changes. It is believed that the presence of these catalysts increases the rate of the autoxidation reaction by increasing the rate of free-radicals necessary to trigger the reaction.

Additionally, lipid-layer based biosensors often include chemical moieties (besides the lipid molecules) that are susceptible to oxidation. For example, the thiol groups and hydrophilic ethoxylate or polyethoxylate (e.g., PEG) linkers used to tether the lipid bilayer to the solid support in ICS biosensors (see above) may undergo oxidation resulting in failure to maintain the lipid layer on the solid support.

Regardless of mechanism, oxidation of the lipid molecule (e.g., formation of an aldehyde) results in structural changes, including hydrolytic cleavage. These changes can affect the structural integrity of a lipid layer of which the oxidized lipid molecule is a part. According to one proposed mechanism, the chemical/structural changes degrade the ability of the lipid molecules in a lipid layer to maintain a close-packed structure. The accumulation of chemical/structural changes due to lipid oxidation results in the structural degradation of the lipid. Ultimately, this oxidative damage prevents the lipid-layer from acting as a barrier for separating two distinct chemical regions. Oxidative damage may also result in desorption of lipid layers from solid supports.

Oxidative damage is a serious problem in the pharmaceutical and food products industry. Both food and pharmaceuticals involve chemical compounds that are susceptible to oxidation and where even minor changes in the chemical/structural properties of these compounds can result severe damage to the function of the product (e.g., loss of efficacy, or flavor). Two primary approaches have been developed for minimizing oxidative damage in pharmaceuticals and foods: (1) adding antioxidant excipients to the product formulation; and/or (2) excluding oxidants from the product formulation.

The ability of a lipid layer to be used as a controllable barrier between two distinct regions provides the foundation for a range of applications, including the sensing of biological molecules. This functionality, however, is highly dependent on the chemical and structural fidelity of the lipid layer. Degradation of the lipid layer due to oxidative damage represents a serious obstacle to achieving improved biosensor performance.

### SUMMARY OF THE INVENTION

The present invention provides methods for manufacture and compositions for lipid layers that exhibit increased stability against oxidative degradation. In addition, the present invention provides methods and materials capable of providing improved storage lifetimes for lipid layer based products, such as biosensors and self-assembled monolayers. In one embodiment, the present invention provides a method for preparing a lipid layer on a solid support with increased resistance to oxidation, said method comprising: providing a solid support; and contacting said solid support with a solution, wherein said solution comprises a lipid layer forming compound and at least one lipid-soluble antioxidant compound at a concentration between about 1 μM and about 10 mM, about 5 μM and about 2 mM, about 25 μM and about 400 μM, or any of the narrower ranges of concentration within the general range of 1 μM to 10 mM. In an embodiment, the method is carried out wherein said solid support is selected from the group consisting of: gold-coated polycarbonate, glass, quartz, and silicon wafer. In another embodiment, the method is carried out wherein the solid support comprises a lipid monolayer. In another embodiment, said lipid monolayer is tethered to the surface of the solid support. In another embodiment, the method further comprises contacting said solid support with an aqueous buffer solution comprising at least one water-soluble antioxidant compound at a concentration between about 0.005% and about 10% w/v, about 0.025% and about 5%, about 0.05% and about 1%, or any of the narrower ranges of concentration within the general range of about 0.005% to about 10% w/v.

In a further embodiment, the method is carried out wherein the lipid layer forming compound is an amphiphilic molecule selected from the group consisting of: phospholipids, glycolipids, thiolipids, bolaamphiphiles, phytanyl lipids, ether lipids, and any combination thereof. In another embodiment, the lipid layer forming compound is an amphiphilic molecule selected from the group consisting of: 1,2-di-O-phytanyl-sn-glycero-3-phosphatidylcholine (DPEPC), 1,2-di-O-phytanyl-sn-glycerol (GDPE), and any combination thereof.

In another embodiment, the method is carried out wherein said lipid soluble antioxidant compound is selected from the group consisting of: vitamin E, tocopherols, tocotrienols, phenols, BHA, BHT, thiols, sulfides, disulfides, sulfoxides, hydroquinones, ascorbyl palmitate, phenylenediamines, gallates, thiocarbamates, and any combination thereof. In addition, the method can be carried out wherein the solution comprises at least two lipid soluble antioxidant compounds selected from the group consisting of: vitamin E, tocopherols, tocotrienols, phenols, BHA, BHT, thiols, sulfides, disulfides, sulfoxides, hydroquinones, ascorbyl palmitate, phenylenediamines, thiocarbamates, gallates, and any combination thereof. In another embodiment, this method is carried out wherein the at least two lipid soluble antioxidant compounds are vitamin E and BHT.

In another embodiment, the method of the present invention can be carried out wherein said method further comprises contacting said support with a barrier compound solution after contacting with the lipid layer forming compound. In one embodiment said barrier compound solution is an aqueous solution, and in one embodiment, said barrier compound solution comprises a film forming polymer. In one another embodiment, said barrier compound is selected from the group consisting of: trehalose, sucrose, mannitol, polyvinylalcohol, cationic polymers, starch, polystyrene sulfonate, polyethylene glycol, and polyethylene oxide. In another embodiment, said method is carried out wherein said barrier compound solution the barrier compound solution comprises trehalose at a concentration between about 0.1% and about 10%, about 0.5% and about 8%, about 1% and about 10%, about 2% and about 10%, or any of the narrower ranges between the general range of 0.1% to 10%.

The present invention also provides a lipid layer on a solid support with increased resistance to oxidation made according to the above-described methods.

In another embodiment, the present invention provides a lipid layer on a solid support with increased resistance to oxidation, wherein said lipid layer comprises a close-packed layer of an amphiphilic molecule, wherein the layer includes at least one lipid soluble antioxidant compound at a concentration between about 0.1 ppm and about 5000 ppm, about 0.5 ppm and about 1000 ppm, about 2.5 ppm and about 250 ppm, about 5 ppm and about 500 ppm, or any other of the narrower ranges in the general concentration range of 0.1 ppm to 5000 ppm. In one embodiment of the lipid layer, the amphiphilic molecule is selected from the group consisting of: phospholipids, glycolipids, thiolipids, bolaamphiphiles, phytanyl lipids and ether lipids. In one embodiment of the lipid layer, the lipid soluble antioxidant comprises one or more compounds selected from the group consisting of: Vitamin E, tocopherols, tocotrienols, phenols, BHA, BHT, thiols, sulfides, disulfides, sulfoxides, hydroquinones, ascorbyl palmitate, phenylenediamines, and gallates. In another embodiment, the lipid layer comprises at least two lipid soluble antioxidant compounds are: vitamin E and BHT. In another embodiment, the lipid layer further comprises a barrier compound solution in contact with the close-packed layer of amphiphilic molecules. In a further embodiment, said barrier compound is selected from the group consisting of: trehalose, sucrose, raffinose, polyvinylalcohol, cationic polymers, starch, polystyrene sulfonate, polyethylene glycol, and polyethylene oxide.

In another embodiment, the invention provides a lipid layer on a solid support with increased resistance to oxidation, wherein the lipid layer is the top layer of a bilayer, wherein the lower layer of the bilayer is attached to the surface of the solid support. In a further embodiment, the lower layer of the bilayer is attached to the surface of the solid support through a linker (i.e., a spacer or tethering compound). In another embodiment, the bilayer comprises an ion channel.

The present invention also provides a method for extending the storage lifetime of a lipid layer on a solid support, said method comprising: providing a solid support comprising a lipid layer; contacting the lipid layer with a barrier compound solution, wherein the barrier compound is selected from the group consisting of: trehalose, sucrose, raffinose, polyvinylalcohol, cationic polymers, starch, polystyrene sulfonate, polyethylene glycol, and polyethylene oxide.

In another embodiment, the method comprises, after contacting the lipid layer with a barrier compound solution, dehydrating the lipid layer to a relative humidity level of less than about 15%. In another embodiment, dehydrating can be carried out by exposing the lipid layer in a chamber to an atmospheric pressure of less than about 200 milliTorr (mTorr) for at least 30 minutes. In another embodiment, dehydration by exposure to low pressure is followed by storage at a relative humidity level of less than about 15%, and typically between about 0.1% and 15%. In other embodiments of this method, the lipid layer can be dehydrated through exposure to a vacuum less than about 150 mTorr, less than about 100 mTorr, or even less than about 50 mTorr. In another embodiment, the vacuum exposure time may be increased to less than about 20 minutes, or even less than about 10 minutes, with an accompanying increase in vacuum resulting in a pressure of less than about 100 mTorr, or less. Similarly, in another embodiment, the dehydrated solid support may be stored at a relative humidity of less than about 12%, 10%, 8%, 5%, or even less than about 2%.

In another embodiment of the method for extending lipid layer storage lifetime, following an initial stage of dehydration about 200 mTorr for at least 30 minutes, the lipid layer is exposed to an atmospheric pressure between about 0.1 and 20 mTorr at 40° C. for at least about 30 minutes followed by storage under inert gas at a relative humidity between about 0.1% and about 15%.

In another embodiment, the method of extending storage lifetime further comprises sealing the lipid layer in a container comprising oxidant barrier material under anaerobic conditions. In one embodiment, the anaerobic conditions comprise a relative humidity of between about 0.1% and about 15%. The method can also be carried out wherein the anaerobic conditions comprise an atmospheric concentration of oxidant molecules less than about 1000 ppb, 100 ppb, or most preferably, less than about 10 ppb, and/or wherein the anaerobic conditions comprise a gas atmosphere selected from the group consisting of: argon, nitrogen, sulfur hexafluoride and carbon dioxide. Further, this method can be carried out wherein the oxidant barrier material is selected from the list consisting of: glass, mylar, high-density polypropylene, aluminum foil, polyvinylidenchloride, polyester, polyamide and cellulose films, and all combination thereof. This method of increasing storage lifetime can also be carried out wherein an oxidant scavenging material selected from the list consisting of: sulfite and bisulfite, finely divided metals, heated metal elements, tannins, carbohydrazides, enzymes including glucose oxidase, and unsaturated organic compounds.

In another embodiment, the above method of increasing storage lifetime can be carried out wherein the lipid layer is the top layer of lipid bilayer, wherein the lower layer is attached to the surface of the solid support. In another embodiment, the lower layer of the bilayer is attached to the surface of the solid support through a linker, and the top and bottom layers each comprise ionophores.

The invention also provides an ion channel sensor with increased oxidative resistance comprising: a solid support with a conducting surface; a lipid bilayer with first and second layers each comprising closely packed amphiphilic molecules, wherein the first layer is attached to the conducting surface of the solid support and comprises a first ionophore, and wherein the second layer comprises a second ionophore, and at least one lipid soluble antioxidant compound at a concentration of between about 0.1 ppm and about 5000 ppm and a plurality of recognition molecules covalently attached to the second ionophores, wherein the recognition molecules are capable of binding to an analyte. In one embodiment, the lipid soluble antioxidant of the ion channel sensor comprises one or more compounds selected from the group consisting of: vitamin E, tocopherols, tocotrienols, phenols, BHA, BHT, thiols, sulfides, disulfides, and sulfoxides. In another embodiment, the ion channel sensor comprises a lipid layer including at least two lipid soluble antioxidant compounds are: vitamin E and BHT. In some embodiments, the lipid soluble antioxidant compound concentration is between about 0.5 ppm and about 1000 ppm, about 2.5 ppm and about 250 ppm, about 5 ppm and about 500 ppm, or any of the narrower ranges in the general concentration range of 0.1 ppm to 5000 ppm.

The invention also provides an ion channel sensor product comprising: an ion channel sensor sealed in an oxidant barrier material container under anaerobic conditions, wherein the ion channel sensor comprises: (a) a solid support with a conducting surface; (b) a lipid bilayer with first and second layers each comprising closely packed amphiphilic molecules, wherein the first layer is attached to the conducting surface of the solid support and comprises a first ionophore, and wherein the second layer comprises a second ionophore, and at least one lipid soluble antioxidant compound at a concentration of about 0.001% to about 5% percent; and (c) a plurality of recognition molecules covalently attached to the second ionophores, wherein the recognition molecules are capable of binding to an analyte, wherein said ion channel sensor product has increased resistance to oxidation. In one embodiment, the ion channel sensor product further comprises a barrier compound in contact with the second layer of the lipid bilayer.

In another embodiment, the ion channel sensor product comprises a dehydrated lipid bilayer with a water activity level of less than about 1.0, 0.75, 0.50, 0.25, 0.10, or even as low as about 0.01. In one embodiment, the water activity level of the dehydrated lipid bilayer is between about 0.01 and about 0.5, or about 0.01 and about 0.2, or any of the narrower ranges in the general water activity level range of 0.01 to 0.5. In another embodiment, the ion channel sensor product comprises a dehydrated lipid bilayer, wherein the dehydrated bilayer has lipid fluidity (as determined by fluorescence photobleaching recovery time) less than about 80%, 60%, 40%, or even about 20% of the lipid fluidity of a fully hydrated bilayer. In another embodiment, the ion channel sensor product comprises a dehydrated lipid bilayer, wherein the diffusion coefficient of lipids in the lipid layer is between about 10% and about 50% of that for a fully hydrated layer. In another embodiment, the invention provides an ion channel sensor product wherein the anaerobic conditions comprise an atmospheric concentration of oxidant molecules less than about 1000 ppb, 100 ppb, or most preferably, less than about 10 ppb, and/or wherein the anaerobic conditions comprise a gas atmosphere selected from the group consisting of: argon, nitrogen, sulfur hexafluoride and carbon dioxide.

In another embodiment, the ion channel sensor product comprises an oxidant barrier material selected from the list consisting of: glass, mylar, high-density polypropylene, aluminum foil, polyvinylidenchloride, polyester, polyamide and cellulose films, and any combination thereof. In another embodiment, the ion channel sensor product comprises an oxidant scavenging material selected from the list consisting of: sulfite and bisulfite, finely divided metals, heated metal elements, tannins, carbohydrazides, enzymes including glucose oxidase, and unsaturated organic compounds

### DETAILED DESCRIPTION OF THE INVENTION

I. Definitions

“Lipid” as used herein refers to any of the class of amphiphilic organic hydrocarbon molecules that include both a hydrophobic and hydrophilic portions, and are substantially soluble in nonpolar organic solvents but substantially insoluble in aqueous solution. Lipids include both naturally occurring and non-natural fats, oils, waxes, sterols, triglycerides, phospholipids, glycolipids, thiolipids, bolamphiphiles, phytanyl lipids and ether lipids.

“Lipid layer” as used herein refers to a two-dimensional assembly of substantially close-packed lipid molecules, including monolayers and bilayers. Lipid layers can include pores (e.g., ionophores) or other inclusion molecules that extend through the layer. Lipid layers can be formed by self-assembly or other non-spontaneous forces. Lipid layers can include two-dimensional assemblies of lipid molecules supported on solids, liquids, or gels. Lipid layers can include lipid assemblies on substantially planar, or substantially non-planar solid supports (e.g., spherical beads or particles).

“Lipid soluble” as used herein refers to the ability of a molecule to be solvated by or dissolved in a phase substantially comprising lipid molecules. For example, lipid soluble molecules are capable of insertion (i.e., inclusion) partially or fully into a lipid layer.

“Water soluble” as used herein refers to the ability of a molecule to be solvated by or dissolved in a phase substantially comprising water molecules (e.g., an aqueous solution).

“Amphiphilic molecule” as used herein refers to a molecule having a hydrophilic head portion and one or more hydrophobic tails. For example, amphiphilic molecules may include a variety of lipid molecules including phospholipids, glycolipids, thiolipids, bolaamphiphiles, phytanyl lipids, and ether lipids.

“Ionophore,” as used herein, refers to a natural or synthetic substance that promotes the passage of ions through lipid barriers in natural or artificial membranes. An ionophore can form an ion-conducting channels or pores in lipid layers or membranes. Ionophores include gramicidin (preferably gramicidin A), band three protein, bacteriorhodopsin, proteorhodopsin, mellitin, alaamethicin, an alamethicin analogue, porin, tyrocidine, tyrothricin, α-haemolysin, and valinomycin.

“Linkers,” “tethers,” or “spacers,” as used herein, refer to chemical compounds or moieties that act to attach a molecule to a solid support, thereby immobilizing it to that support. Attachment via linkers, tethers, or spacers can occur via the formation of covalent, ionic, hydrogen bonding, or some combination of any of these types of interactions between the surface of the solid support and the molecule to be immobilized (e.g., a lipid molecule).

“Solid support” as used herein refers to any solid substrate material with at least one surface to which a lipid layer can adhere. Solid supports can include glass, quartz, silicon, polymers (e.g., polycarbonate, polystyrene) metals, and metal coatings on other substrates, and gels. Solid supports generally provide a surface to which lipid layers can be bound covalently or non-covalently through direct or indirect binding. Additionally, solid supports can provide functionality (e.g., as electrodes) when incorporated into sensors.

“Oxidative damage” or “oxidative degradation” as used herein refers to the detrimental effects on the structure and/or function of a molecule or molecular assembly (e.g., a lipid layer) caused by an oxidation reaction.

“Stability against” or “increased resistance to” oxidative damage (or degradation) as used herein refers to the decrease in the effects of oxidative damage (or degradation) conferred by a protective measure (e.g., inclusion of antioxidant) relative to the level of oxidative damage (or degradation) observed for a control without that protective measure.

“Antioxidant compound” or “antioxidant” as used herein refers to any compound capable of substantially inhibiting or preventing an oxidation reaction from occurring, via any mechanism, direct or indirect, catalytic or stoichiometric. For example, antioxidant compounds may include vitamin E, tocopherols, tocotrienols, BHA, BHT, thiols (e.g., DTT), sulfides, disulfides, sulfoxides (e.g., DMSO), hydroquinones, ascorbyl palmitate, phenylenediamines, thiocarbamates, and gallates.

“Barrier compound” as used herein refers to all water soluble compounds capable of forming an airtight barrier on top of the lipid bilayer, including sugars, salts and all film forming polymers. For example, barrier compounds may include trehalose, sucrose, mannitol, raffinose, polyvinyl alcohol, cationic polymers, starch, polystyrene sulfonate, polyethylene glycol, polyethylene oxide.

“Water activity level,” “water activity value,” or “A_{w}” as used herein is a measure of the amount of water associated with a lipid layer that is related to relative humidity (“RH”) according to the equation, A_{w}=RH/100. For example, the water activity value of a dehydrated lipid layer system is less than 1, and approaches 0 in the driest systems.

II. Compositions and Methods of the Invention

The present invention provides lipid layers and associated lipid layer based products (e.g., sensors, coatings, monolayers) with increased stability to oxidative degradation with resulting increased storage lifetime and functional utility. Specifically, the embodiments for oxidative protection of lipid layers disclosed herein include: (1) antioxidant inclusion in the lipid layer; (2) coating the lipid layer with oxidant barrier compounds; (3) storage under inert (i.e., non-oxidizing) gases; (4) storage with oxidant scavengers and catalysts; (5) storage in oxidant barrier packaging; and (6) any combination of each of these different methods.

Structure and Function of Lipid Molecules and Lipid Layers

The present invention can be applied generally for oxidative protection to any layer assembly comprising lipid molecules. It is well-known in the art that lipid molecules can be assembled into a variety of layer structures depending on conditions. Typically, the layers formed by lipids are close-packed, meaning that the long axes of the lipid molecules aligned in the same direction with no space between adjacent molecules (e.g., hexagonal close-packing). Perhaps the best known example of a lipid layer structure is the phospholipid bilayer that constitutes cellular membranes. The phospholipids in membranes are long amphiphilic molecules that have a hydrophilic “head” at one end of the long axis, and a hydrophobic “tail” at the other end. This amphiphilic structure encourages the phospholipids to form the well-known structure of two close-packed layers (i.e., a bilayer).

Amphiphilic molecules include any of the well-known types of surfactant molecules such as cationic (e.g., quaternary ammonium salts), anionic (e.g., organosulfonate salts), zwitterionic (e.g., phosphatidyl cholines, phosphatidyl ethanolamines), non-ionic (e.g., polyether materials), and membrane spanning lipid. Amphiphilic molecules useful with the present invention include phospholipids, glycolipids, thiolipids, bolaamphiphiles, phytanyl lipids, and ether lipids. Particularly useful phytanyl lipids include: 1,2-di-O-phytanyl-sn-glycero-3-phosphatidylcholine (DPEPC), and 1,2-di-O-phytanyl-sn-glycerol (GDPE). Lipid layers of the present invention may include mixtures of amphiphilic molecules including any combination of lipid molecules selected from the list consisting of phospholipids, glycolipids, thiolipids, bolaamphiphiles, phytanyl lipids, and ether lipids.

In some embodiments of the present invention, amphiphilic molecules in lipid layers include chemical groups such that they can be crossed linked. Typical cross-linkable moieties include vinyl, methacrylate, diacetylene, isocyano, or styrene groups located either in the head, or in the hydrophobic tail of the amphiphilic molecule. Preferably, the cross-linkable groups are connected to the amphiphilic molecule through a spacer group such as is described in Fukuda et al., J. Amer. Chem. Soc. 108, 2321-2327 (1986).

Inclusions in Lipid Layers

Despite being close-packed, other atoms or molecules can insert (i.e., an inclusion) into a lipid layer. Depending on the structure of the lipid layer and the inclusion, the inclusion can (1) extend completely through the layer; (2) extend partially into the layer; or (3) be completely inserted in the layer. The insertion of these inclusions can occur during or after lipid layer formation. Inclusions can range from single atoms to large assemblies of polypeptides (e.g., ion channels, receptor proteins, etc.).

The ability of lipids to form layers with or without inclusion molecules makes them useful in a wide range of applications where a molecular “boundary” or “barrier” is desirable. Typically, applications seek to use lipid layers to separate two solutions with different chemical potential (e.g., micelles, or solutions with different osmotic potential inside and outside a cell membrane).

Supported Lipid Layers

In one embodiment, the present invention provides compositions and methods for oxidative protection of lipid layers on solid supports (i.e., “supported lipid layers”). Solid supports can include solids of any shape and size that include a surface large enough to support a lipid layer. This can include spherical (e.g., beads), cylindrical (e.g., probes, capillaries), and planar supports (e.g., glass slides, electrodes).

Supported lipid layers useful with the present invention can be manufactured using a range of methods. Typically, they are produced by forming or assembling the lipid on a surface of the solid support. For example, a solution containing the desired lipid molecules can be applied to the surface of the solid support under conditions that promote the spontaneous assembly of the lipid layer. Conditions for spontaneous assembly of lipid layers on solid supports are well-known in the art, or can be determined through routine experimentation. Alternatively, supported lipid layers can be prepared by forming the lipid layer on the surface of a solution and then transferring this preformed lipid layer to the surface of a solid support.

In one embodiment, the present invention provides a method for preparing a lipid layer on a solid support with increased resistance to oxidation, said method comprising: providing a solid support; and contacting said solid support with a solution, wherein said solution comprises a lipid layer forming compound and at least one lipid-soluble antioxidant compound at a concentration of about 1 μM to about 10 mM, about 5 μM to about 2 mM, about 25 μM to about 400 μM, or any of the narrower ranges of concentration within the general range of 1 μM to 10 mM.

Supported lipid layers can be attached to the surface of the solid support through physical adsorption or chemical bonding. Physical bonding can be facilitated by any of the well-known non-covalent bonding interactions (e.g., hydrophobic, van der Waals, hydrogen bonding, etc.) and can include indirect interactions with the solid support through an intervening liquid layer. For example, a supported lipid layer can be physically adsorbed to a thin layer of water molecules that are in turn physically adsorbed to the surface of the solid support.

A range of linkers, tethers, or spacers are well-known in the art for attaching a lipid molecule to the surface of a solid support. For example, linkers or spacer groups useful with the present invention include, but are not limited to, compounds selected from the group consisting of alkyl, alkyl amides, alkyl esters, alkyl carbamates, alkyl carbonates, oligomers of alkylidene glycol (such as ethylene glycol), combinations of oligomers of ethylene glycol with amides, esters or carbamates, and oligopeptides.

The linker or spacer groups useful with the present invention can be hydrophilic (having a tendency to bind or absorb water) or hydrophobic (antagonistic to water and incapable of dissolving in water). For example, spacer groups of the present invention can be selected from the group consisting of saturated or unsaturated C₁₋₈ alkyl, saturated or unsaturated C₃₋₇ cycloalkyl, aryl, aralkyl, heteroaryl, and saturated or unsaturated C₂₋₆ heterocycle; C₁₋₈ alkylamides, C₁₋₈ alkylesters, C₁₋₈ alkylcarbamates, C₁₋₈ alkylcarbonates, oligomers (e.g., n=2-10) of alkylidene glycol (such as ethylene glycol), combinations of oligomers of ethylene glycol with amides, esters or carbamates, and oligopeptides, where in all rings or chains optionally bear one or more desired substituents such as halogen, hydroxy, C₁₋₄ alkoxy, carboxy, cyano, nitro, sulfonamido, sulfonate, phosphate, amino and substituted amino. In some embodiments the spacer group is a C₁₋₈ alkyl, an oligomer of alkylideneglycol, or oligomers of ethylene glycol with amides, esters or carbamates.

Generally, these linkers, spacers, or tethers comprise chemical groups that result in immobilization through the formation of covalent, ionic, hydrogen bonding, or some combination of any of these types of interactions between the surface of the solid support and the lipid layer. The groups can be attached to a lipid molecule, the solid support, or both. Attachment of chemical linker groups to the lipid layer can be before or after the lipid layer is formed. For example, modified lipid molecules can be used to form the lipid layer, wherein the modification (e.g., an amine group) allows the lipid layer, once formed, to form a covalent attachment with a group on the surface of the solid support. Chemistry for modifying solid supports with linkers, spacers, or tethers for further attachment of chemical compounds (e.g., lipid molecules, proteins, nucleic acids) are well-known in the art. For example, a wide range of amine modified silane compounds (e.g., aminopropyl silane) are available for modifying glass and other silica-based supports. In another embodiment of the present invention, one layer of a lipid bilayer is attached covalently to the surface of a solid support through a long chemical spacer or tether attached to many of the lipid molecules in the layer proximal to the support (i.e., the “lower” layer). See e.g., Knoll, et al., J Biotechnol. 74, 137-58 (2000); and Krishna, et al., Langmuir 17, 4858-4866 (2001). The spacer also creates a liquid filled region (also referred to herein as a “reservoir”) between the surface of the solid support and the surface of the lower lipid layer of the bilayer. In one embodiment, the surface of the solid support is gold, and the spacer includes a thiol group capable of bonding to the gold surface. This tethering via the spacer effectively immobilizes the lipid bilayer, a key requirement in many applications for supported lipid layers. For a more detailed description of tethering to gold surfaces through thiol groups see, e.g., Raguse, et al., Langmuir 14, 648-659 (1998).

Oxidative Protection of Lipid Layer Based Biosensors

Lipid layers find utility in numerous applications typically as a material or component that provides a boundary between two aqueous solutions. In particular, lipid layers which can be selectively permeable (e.g., bilayer, membranes) find use in sensing devices where movement of molecules across a barrier creates a signal. Biosensors are one form of sensing device that is used to detect biological molecules, typically a receptor ligand in a solution. One form of biosensor that incorporates lipid layers are ion channel switch (ICS) biosensors. ICS are described in U.S. Pat. Nos. 5,874,316; 5,234,566; 5,443,955; 5,741,409, 5,401,378; 5,637,201; 5,753,093; 5,783,054; 6,316,273; 6,451,196; 6,573,109; and 5,741,712; and published PCT application WO 98/55853; each of which is hereby incorporated by reference herein in its entirety.

Like all sensors that incorporate lipid layers in their design, the performance of an ICS biosensor diminishes as the lipid molecules in the bilayer oxidize and the first or second layer degrades. The loss of performance correlates with exposure to oxidants, most commonly molecular oxygen or ozone in the atmosphere. Loss of performance of a biosensor is easily quantified by using the sensor measuring an identical biological analyte sample before and after the sensor is exposed to an oxidant. Oxidative damage to the lipid layer correlates directly with decreased performance of the biosensor. Consequently, strategies for minimizing oxidative damage to lipid layers can be screened in a facile manner using lipid based biosensors such as ICS biosensors. The present invention provides compositions and methods that maintain the performance of sensors with lipid layers in the presence of oxidants, and extend the storage lifetime of these sensors.

Lipid Monolayer Based Applications

The compositions and methods for oxidative protection of lipid layers are applicable to monolayers, such as SAMs, as well as bilayers (and more complex lipid layer assemblies). Lipid monolayers immobilized on solid supports can also serve as components in sensors, coatings, or nanoscale electronic devices (e.g., organic thin-film transistors). For example, self-assembled monolayers (SAMs) with tethered proteins or enzymes have been used as amperometric or coulometric sensing devices or in surface plasmon resonance (SPR) sensor.

Assessment of Oxidative Damage and Methods for Screening Oxidative Protection Strategies

The present invention provides compositions and methods to minimize the detrimental effects of oxidation (i.e., oxidative damage) on lipid layers. In order to analyze various compositions and strategies for preventing oxidative damage, the present invention provides methods based on measuring oxidative effects on lipid layer based biosensors. In another embodiment, the relative performance of an ion-channel biosensor comprising a lipid bilayer with ion channel inclusions, is measured using impedance spectroscopy. For a review of impedance spectroscopy methods, see, e.g., Janshoff et al., “Applications of impedance spectroscopy in biochemistry and biophysics,” Acta Biochim Pol., 43(2):339-48 (1996). Changes in biosensor performance in the presence of oxidant are then monitored over time following treatment with antioxidant(s) or other methods for excluding oxidant (e.g., barrier coatings).

An unexpected discovery disclosed herein is that concentrations of ozone on the order of about 50 ppb, which is similar to ambient concentrations of ozone in urban environments, will damage the performance of dehydrated ICS biosensors. Thus, although dehydration is often used to protect lipid layer based devices from solution-phase oxidizing agents, this treatment can increase susceptibility to oxidative damage from oxidizing gases, even at sub-ambient concentrations.

In another embodiment of the present invention, dehydrated ICS biosensors are screened for improved performance following exposure to the oxidant ozone for about 15 minutes at an ozone concentration of about 50-100 ppb. After rehydration, biosensor performance parameters are measured. Even at these relatively low levels of ozone exposure while in a dehydrated state, the performance parameters of the ICS biosensors are greatly diminished. As described below, the present invention provides methods (e.g., exposure to an antioxidant solution) that protect against the performance degrading effects of ozone exposure, and exposure to other oxidants.

Alternatively, measurement of oxidative damage and screening of methods to attenuate that damage can be based on the relative performance characteristics of other types of lipid layer based sensors. In some embodiments, any of the well-known chemical or physical methods of detecting chemical or structural changes to the lipid molecules or the overall lipid layer can be used to assess oxidative damage and methods for preventing this damage.

Antioxidant Inclusion in Lipid Layers

The present invention provides lipid layers with antioxidant compounds inclusions that have increased stability against oxidative damage. This increased stability manifests as increased storage lifetime and functionality for products based on lipid layers, such as biosensors. Although the ability of antioxidant compounds to inhibit oxidation reactions is well-known, the present invention discloses the powerful protective effect of antioxidant inclusions on lipid layers. Antioxidant inclusions are antioxidant molecules inserted in the lipid layer either partially or completely. Without being bound by theory, the insertion of molecules into a lipid layer is similar to solvation in a solution. In order to insert, the molecule to be include should be substantially soluble in lipids (i.e., lipid soluble).

Generally, lipids are substantially soluble in organic, non-polar solvents (e.g., saturated and unsaturated hydrocarbons, fats, oils) and substantially insoluble in aqueous (and more polar, hydrophilic) solvents. Thus, inclusion of antioxidant compounds in lipid layers occurs preferentially for those antioxidant compounds that are substantially soluble in organic, non-polar solvents. Table 1 lists several exemplary antioxidant compounds and their relative solubilities. Generally, those antioxidants that exhibit some solubility in oil are lipid soluble and can insert as antioxidant inclusions in lipid layers in accordance with the present invention. Thus, in one embodiment, the present invention provides a lipid layer comprising at least one lipid soluble antioxidant inclusion selected from the list consisting of: α-tocopherol acetate; d-α-tocopherol (nat); d-α-tocopherol (syn); ascorbyl palmitate; butylated hydroxyanisole (BHA); butylated hydroxytoluene (BHT); d-tocopherol (syn); dilauryl thiodipropionate; dodecyl gallate; ethoxyquin; gallic acid; γ-tocopherol (syn); gossypol; hydroquinone; 4-hydroxymethyl-2,6-di-tert-butylphenol; lecithin; α-lipoic acid (Na salt); α-naphthol; β-naphthoquinone; nordihydroguaiaretic acid; octyl gallate; phenols (m- and p-diphenols); sulfur dioxide; thioglycolic acid; thiolactic acid; thiosorbitol; and tocopherols.

In one embodiment, the list of antioxidants useful as inclusion molecules in lipid bilayers according to the compositions and methods of the present invention includes: vitamin E, tocopherols, tocotrienols, phenols, BHA, BHT, thiols, sulfides, disulfides, and sulfoxides, including DMSO. In another embodiment, the antioxidants useful as inclusion molecules in self-assembled monolayers includes compounds such as vitamin E, tocopherols, tocotrienols, phenols, BHA, BHT, thiols, sulfides, disulfides, but does not include small solvent compounds such as DMSO and DMF.

Generally, antioxidant compounds exhibit slightly different chemical and physical properties and in some cases it can be advantageous to include a mixture of any two or more different antioxidant compounds selected from the list in Table 1 in a lipid layer. In another embodiment, the present invention provides a lipid layer comprising at least two lipid soluble antioxidant inclusions selected from the list consisting of: α-tocopherol acetate; d-α-tocopherol (nat); d-α-tocopherol (syn); ascorbyl palmitate; butylated hydroxyanisole (BHA); butylated hydroxytoluene (BHT); d-tocopherol (syn); dilauryl thiodipropionate; dodecyl gallate; ethoxyquin; gallic acid; γ-tocopherol (syn); gossypol; hydroquinone; 4-hydroxymethyl-2,6-di-tert-butylphenol; lecithin; α-lipoic acid (Na salt); α-naphthol; β-naphthoquinone; nordihydroguaiaretic acid; octyl gallate; phenols (m- and p-diphenols); sulfur dioxide; thioglycolic acid; thiolactic acid; thiosorbitol; phenylenediamines and tocopherols. In one particularly useful embodiment, the invention provides a lipid layer comprising vitamin E and BHT inclusions.

TABLE 1
Selected antioxidants

| | Solubility | | |
|---|---|---|---|
| Compound | In water | In alcohol | In oil |
| Acetone sodium bisulfite | Yes | No | No |
| Acetylcysteine | Yes | Yes | No |
| α-tocopherol acetate | No | Yes | Yes |
| d-α-tocopherol (nat) | No | Yes | Yes |
| d-α-tocopherol (syn) | No | Yes | Yes |
| Ascorbic acid | Yes | Yes | No |
| Ascorbyl palmitate | Yes | Yes | Yes |
| Butylated hydroxyanisole | No | Yes | Yes |
| Butylated hydroxytoluene | No | Yes | Yes |
| Calcium ascorbate | Yes | Yes | — |
| Calcium bisulfite | Yes | — | — |
| Calcium sulfite | Yes | Yes | — |
| Cysteine | Yes | Yes | No |
| Cysteine HCl | Yes | Yes | No |
| D-tocopherol (syn) | No | Yes | Yes |
| Dilauryl thiodipropionate | No | Yes | Yes |
| Dithiothreitol | Yes | Yes | No |
| Dodecyl gallate | No | Yes | Yes |
| Ethoxyquin | — | — | Yes |
| Ethyl gallate | Slightly | Yes | No |
| Gallic acid | Yes | Yes | Yes |
| γ-tocopherol (syn) | No | Yes | Yes |
| Glutathione | Yes | — | — |
| Gossypol | No | Yes | Yes |
| Hydroquinone | Yes | Yes | Yes |
| 4-Hydroxymethyl-2,6-di-tert- | Yes | Yes | Yes |
| butylphenol | | | |
| Hypophosphorus acid | Yes | — | — |
| Isoascorbic acid | Yes | — | — |
| Lecithin | Yes | Yes | Yes |
| α-lipoic acid (Na salt) | (Yes) | — | Yes |
| Monothioglycerol | Yes | Yes | — |
| α-naphthol | Yes | Yes | Yes |
| β-naphthoquinone | | | |
| Nordihydroguaiaretic acid | No | Yes | Yes |
| Octyl gallate | No | Yes | Yes |
| Phenols (m- and p-diphenols) | | | |
| Potassium metabisulfite | Yes | No | No |
| Propyl gallate | Slightly | Yes | Slightly |
| Sesamol | — | — | — |
| Sodium ascorbate | Yes | Yes | No |
| Sodium bisulfite | Yes | Slightly | No |
| Sodium formaldehyde | | | |
| sulphoxylate | Yes | Slightly | — |
| Sodium metabisulfite | Yes | Slightly | — |
| Sodium sulfite | Yes | No | No |
| Sodium thiosulfate | Yes | No | — |
| Sulfur dioxide | Yes | Yes | Yes |
| Tannic acid | Yes | | |
| Thioglycerol | Yes | Yes | — |
| tert-Butyl-hydroquinone | — | — | — |
| Thioglycolic acid | Yes | Yes | Yes |
| Thiolactic acid | Yes | Yes | Yes |
| Thiosorbitol | Yes | Yes | Yes |
| Thiourea | Yes | Yes | No |
| Tocopherols | — | — | Yes |
| 2,4,5-trihydroxy-butyrophenone | — | — | — |

The present invention provides methods for inclusion of antioxidant compounds in lipid layers. In one embodiment, antioxidant molecules are included prior to, or during, the formation of the lipid layer. In this embodiment, the antioxidant compound is dissolved in the lipid layer forming solution, at a concentration of about 1 μM to about 10 mM, about 5 μM to about 2 mM, about 25 μM to about 400 μM, or any of the narrower ranges of concentration within the general range of 1 μM to 10 mM.

In some embodiments, mixtures of two or more antioxidants are used in an “antioxidant cocktail.” Where such mixtures of antioxidants are used, the concentrations of each antioxidant may be varied independently within the general ranges described for the individual antioxidants. Thus, each antioxidant component can be included at a different concentration within the ranges of about 1 μM to about 10 mM, about 5 μM to about 2 mM, about 25 μM to about 400 μM, or any of the narrower ranges of concentration within the general range of 1 μM to 10 mM. For example, in one embodiment, the lipid layer forming solution includes an antioxidant cocktail comprising about 20 μM to about 500 μM vitamin E and about 4 μM to about 100 μM BHT. In one particular embodiment, the antioxidant cocktail comprises about 100 μM vitamin E and about 20 μM BHT.

Alternatively, an antioxidant solution can be added to a lipid layer following formation of the lipid layer. In this embodiment, the antioxidant solution is allowed to contact the lipid layer for a set incubation period wherein the antioxidant molecules in the solution undergo a partitioning into the lipid layer phase. In this embodiment, it is preferred to use antioxidant molecules soluble in both aqueous and lipid environments. By contacting the lipid layer with an aqueous antioxidant solution that is relatively insoluble with the lipid layer, the lipid soluble antioxidant molecules favor solvation into the lipid layer. The incubation period where the antioxidant solution contacts the lipid layer depends on the antioxidant molecule and/or the fluidity of the lipid layer, which in turn depends on the specific type of lipid molecules, temperature, and hydration of the layer. Thus, in an embodiment for a highly fluid lipid layer that exhibit rapid incorporation of molecules in solution, the incubation period can be short, e.g., about 30 seconds to about 15 minutes. In an embodiment with a less fluid lipid layer (e.g., less hydrated), the incubation period can be considerably longer, ranging from about 1-2 hours, about 6-12 hours, 12-24 hours, or even longer.

The present invention provides lipid layers that exhibit greater oxidative protection regardless of the method of formation. As disclosed herein, lipid layer compositions comprising antioxidant inclusion molecules at a concentration range of between about 0.1 ppm and about 5000 ppm relative to lipid molecules exhibit improved resistance to oxidation. In particular embodiments of the present invention, the concentration range of antioxidant inclusion molecules can include about 0.5 ppm to about 1000 ppm, about 2.5 ppm to about 250 ppm, about 5 ppm to about 500 ppm, about 50 ppm to about 200 ppm, or any other of the narrower ranges in the general concentration range of 0.1 ppm to 5000 ppm. Of course, mixtures of antioxidants are used (e.g., an antioxidant cocktail), the concentrations of each antioxidant in the lipid layer will can differ, but each concentration will fall within the general ranges described for the individual antioxidants.

In another embodiment, lipid layers with increased resistance to oxidation can be prepared using water soluble antioxidants (e.g., ascorbic acid) in the buffer solutions used during the preparation and use of the lipid layers. Water soluble antioxidants useful with this embodiment include, but are not limited to, those listed in Table 1: acetone sodium bisulfite; acetylcysteine; ascorbic acid; ascorbyl palmitate; calcium ascorbate; calcium bisulfite; calcium sulfite; cysteine; cysteine HCl; dithiothreitol; ethyl gallate; gallic acid; glutathione; hydroquinone; 4-hydroxymethyl-2,6-di-tert-butylphenol; hypophosphorus acid; isoascorbic acid; lecithin; α-lipoic acid (Na salt); monothioglycerol; α-naphthol; potassium metabisulfite; propyl gallate; sodium ascorbate; odium bisulfite; sulphoxylate; sodium metabisulfite; sodium sulfite; sodium thiosulfate; sulfur dioxide; tannic acid; thioglycerol; thioglycolic acid; thiolactic acid; thiosorbitol; and thiourea. In some embodiments, the water soluble antioxidants will be added in solution to the lipid layer prior to its formation, e.g., in the second layer solution used in forming a lipid bilayer biosensor. In another embodiment, aqueous solutions comprising one or more water-soluble antioxidants may be contacted with the surface of a lipid layer after its formation, or upon rehydration of a previously dehydrated lipid layer.

Thus, the present invention also provides a method for preparing a supported lipid layer with increased resistance to oxidation comprises contacting said solid support, prior to or after formation of the lipid layer, with an aqueous buffer solution comprising at least one water-soluble antioxidant compound at a concentration between about 0.005% and about 10% w/v, about 0.025% and about 5%, about 0.05% and about 1%, or any of the narrower ranges of concentration within the general range of about 0.005% to about 10% w/v.

Thus, the present invention provides numerous combinations of supported lipid layers with antioxidant inclusion molecules. For example, in one particular embodiment, the present invention provides a gold-coated polycarbonate support coated with a phospholipid bilayer that comprises vitamin E and BHT at concentrations of 100 ppm and 20 ppm, respectively.

Methods for determining concentration of antioxidant compounds are well-known in the art (e.g., GC-MS, HPLC, NMR, etc.). The final antioxidant inclusion concentration will be directly proportional to the starting concentration of antioxidant compound in the lipid layer forming solution, and/or the conditions for forming the lipid layer (or the conditions for adding the antioxidant to a pre-formed layer). Although antioxidant concentration can vary, lipid layer compositions with antioxidant inclusion concentrations in the general range of about 0.1 ppm to about 5000 ppm will exhibit the functional properties of increased stability to oxidative damage relative to unmodified lipid layers when exposed to oxidants (e.g., oxygen, ozone, etc.) in screening methods like those described in detail below.

Dehydration of Lipid Layers

The present invention also includes methods for increasing the storage lifetime of lipid layer based products by dehydration of the product prior to storage. Dehydration reduces the fluidity of lipid layers and thereby affords protection against defect formation that results in desorption of supported layers. Techniques for safely dehydrating lipid layers are well-known in the art and can be used in accordance with the methods of the present invention. For example, it is known that the drying of lipid membranes should be conducted in such a manner so as to prevent damage to the membrane through the formation of ice crystals, rapid outgassing of water vapor, or other processes detrimental to the integrity of the bilayer.

Without being bound by theory, it is recognized that in some cases, the presence of residual water serves to maintain the fluidity of lipid layer while it is in the dehydrated state. The residual water also acts to maintain a defect free bilayer upon rehydration. Certain additives, for example sugars including trehalose, can serve to replace bound water associated with the polar lipid head groups whilst maintaining a defect free lipid bilayer. Thus, as described in greater detail below, trehalose and other sugars can be used in “barrier compound coatings” to preserve the integrity of lipid bilayers during drying and rehydration steps.

In the case of a supported lipid bilayer it is also recognized by those of skill that some water remains in the reservoir region between the bilayer and the solid support even after rigorous drying. See, e.g., Crowe et al., “Stabilization of dry phospholipid bilayers and proteins by sugars,” Biochem. J., 242:1 (1987). Thus, the present invention provides supported lipid bilayers (e.g., ICS biosensors) that are dehydrated but still retain water in the reservoir region. These dehydrated bilayers exhibit increased storage lifetime due to dehydration and are easily hydrated for use after storage.

Determination of degree of hydration (or dehydration) may be necessary for determining optimal lipid layer storage conditions. Several methods for qualitative and quantitative determination of the relative hydration (or extent of dehydration) of a lipid bilayer are well-known in the art. For example, Reflection-Adsorption Infrared Spectroscopy (“RAIRS”) (see e.g., Mateo-Marti et al., “Self-assembled monolayers of peptide nucleic acids on gold surfaces: a spectroscopic study,” Langmuir 21(21):9510-7 (2005)), mass spectrometry, neutron reflection spectroscopy, are techniques known to those of skill for determining lipid layer hydration.

According to the present invention, dehydration or drying of a supported lipid layer can be carried out by placing the solid support in a chamber and reducing the pressure reduced from atmospheric pressure (i.e., ˜760 mTorr) to less than about 200 mTorr (i.e., “low vacuum”) at a rate so as to prevent the formation of defects in the lipid layer. The supported lipid layer is held under these “low vacuum” conditions for at least about 30 minutes, after which the vacuum is released. Subsequently, in one embodiment, the lipid layer is stored under an inert gas (e.g., nitrogen) and relative humidity less than about 20%, about 15%, about 10%, or even less than about 5%. Similarly effective variations on this general method may also be used. For example, one can apply a higher vacuum (e.g., about 100 mTorr, 50 mTorr, or less) for a shorter time period (e.g., less than about 25, about 20, about 15, or even less than about 10 minutes).

In another embodiment, a second stage of drying may be applied where the relative atmospheric pressure is further reduced to between 0.1 and 20 mTorr (i.e., “high vacuum”), and the temperature is increased to over 40 C. Lipid bilayers can be held under these high vacuum conditions at elevated temperature for about 30 minutes or more, followed by an additional period wherein the temperature is reduced to room temperature. Following such a two-stage drying process the vacuum is released and the lipid layers stored under an inert gas at a relative humidity level of between about 0.1% and about 15%.

Typically, even after dehydrating a lipid layer in one or two stages, as described above, some residual water remains associated with the dried layer. This residual water is not in the form of liquid water, but rather water molecules tightly bound to the hydrophilic regions of the lipid bilayer. Thus, in another embodiment, the dehydration of the lipid layers of the present invention may be characterized in terms of the water activity value “A_{w}”. Water activity is related to relative humidity (“RH”) according to the equation, A_{w}=RH/100. Generally, the water activity value of any dehydrated lipid layer system is less than 1, and approaches 0 in the driest systems (see, e.g., Binder et al., Chem. Phys Lett., 304: 324-335 (1999)).

Thus, in another embodiment of the present invention, the storage lifetime of a lipid layer can be increased by dehydrating the layer to a water activity value of less than about 1.0, less than about 0.5, less than about 0.4, less than about 0.3, less than about 0.2, or even less than about 0.1. Following drying, the water activity value for a lipid layer can be maintained by storage at a relative humidity level of between about 0.1% and about 15%.

Lipid layer “fluidity” represents another characteristic that may be determined and modified in accordance with the present invention in order to provide lipid layers with increased storage lifetime. The fluidity of lipid layer corresponds to the degree of hydration of the layer. Lipid layer fluidity can be measured by well-known techniques such as Fluorescence Recovery after Photobleaching (“FRAP”) (for a review of FRAP see e.g., Meyvis et al., “Fluorescence recovery after photobleaching: a versatile tool for mobility and interaction measurements in pharmaceutical research,” Pharm Res. 16(8):1153-62 (1999)). This method involves the addition of a fluorescent dye to the lipid bilayer followed by photobleaching of a small area of the lipid bilayer with a high-powered laser. The rate and extent of fluorescence recovery in the bleached area allows one to calculate the fluidity of the lipid membrane. Fully hydrated lipid membranes typically exhibit fluorescence recoveries of greater than 90% with diffusion coefficients of 2×10⁻⁶ cm²/sec to over 15×10⁻⁶ cm²/sec (See e.g., Wagner et al., Biophys. J., 79, 1400 (2000)). Following drying both the rate and extent of lipid fluidity fall as the number of water molecules associated with the membrane is reduced. Lipid membranes dried in the presence of sugars including trehalose exhibit further reductions in lipid fluidity upon drying relative to lipid bilayers dried in the absence of trehalose and other sugars.

Thus, in one embodiment, the present invention provides dehydrated lipid layers, and/or lipid layer based products (e.g., biosensors) with increased storage lifetime, wherein the fluidity of the lipid is such that the fluorescence recovery following photobleaching is less than about 80%, less than about 60%, less than about 40%, less than about 30%, or even less than about 20% of that of a fully hydrated lipid bilayer.

In another embodiment, the present invention provides, dehydrated lipid layers and/or lipid layer based products with increased storage lifetime, wherein the dehydrated lipid layer has a diffusion coefficient of lipids in a dried lipid bilayer less than about 50%, less than about 30%, less than about 20%, or even less than about 10% that of a fully hydrated lipid bilayer.

Although dehydration can increase storage lifetime of lipid layer based products, it does not fully protect them from oxidative damage. Indeed, under some conditions, susceptibility to oxidative damage (particularly due to ozone exposure) increases when the lipid layer is dehydrated. Consequently, in accordance with the present invention, dehydration of lipid layers should be used in combination with other techniques for reducing oxidative damage such as: addition of antioxidant inclusions, use of barrier compound coatings, storage under an inert atmosphere, and storage in packaging capable of acting as an oxidant barrier.

Barrier Compound Coatings

In accordance with the present invention, barrier compound coatings can be used in combination with lipid layers with antioxidant inclusions thereby providing increased protection from oxidative damage. Thus, the present invention also provides a method for extending the storage lifetime of a lipid layer on a solid support, said method comprising: providing a solid support comprising a lipid layer; contacting the lipid layer with a barrier compound solution, wherein the barrier compound is selected from the group consisting of: trehalose, sucrose, raffinose, polyvinylalcohol, cationic polymers, starch, polystyrene sulfonate, polyethylene glycol, and polyethylene oxide.

Barrier compounds useful with the present invention include all compounds that form an air-tight barrier when applied as an aqueous solution on top of a lipid layer. Among the barrier compounds most useful with the present invention are sugars (e.g., trehalose, sucrose, or raffinose), salts, and all film-forming polymers (e.g., enteric polymer coatings such as cellulose acetate phthalate, hydroxypropyl methylcellulose, and polyvinyl acetate phthalate). In one embodiment, the barrier compound solution comprises a barrier compound selected from the group consisting of: trehalose, sucrose, raffinose, polyvinylalcohol, cationic polymers, starch, polystyrene sulfonate, polyethylene glycol, and polyethylene oxide. In one particularly useful embodiment, the barrier compound solution comprises trehalose at a concentration between about 0.1% and about 10%, about 0.5% and about 8%, about 1% and about 10%, or any of the narrower ranges between the general range of 0.1% to 10%.

The barrier compound solution can be applied to the lipid layer before or after the lipid layer has been dehydrated. For example, in one embodiment a lipid bilayer is contacted with of a 10% trehalose solution, followed by controlled drying under vacuum such as to leave a glassy trehalose coating free from cracks or defects covering the lipid bilayer. In another embodiment the barrier solution can be applied to a dehydrated lipid bilayer by spray coating with a barrier compound or solution thereof. Thus, the method can further comprise a step of storing the solid support at a relative humidity level between about 0.1% and about 15% after contacting the lipid layer with a barrier compound solution. See e.g., Crowe et al., “Stabilization of dry phospholipid bilayers and proteins by sugars,” Biochem. J., 242:1 (1987).

Thus, the present invention provides lipid layers with or without antioxidant inclusion molecules, but with a barrier coating, and in a further embodiment, dehydrated. For example, in one particular embodiment, the present invention provides a gold-coated polycarbonate support coated with a phospholipid bilayer that comprises vitamin E and BHT at concentrations of 100 ppm and 20 ppm, respectively, and which is coated with a 10% trehalose, and which is dehydrated such that the fluidity of the lipid layer is about 30% of a fully hydrated lipid layer when determined by FRAP.

Storage Under Inert Atmosphere

In another embodiment of the present invention, further protection of lipid layers from oxidation is achieved by storage of the lipid layer under an atmosphere of one or more inert (i.e., non-oxidizing) gases. Thus, additional oxidative protection can be achieved by sealing the lipid layers of the present invention in a container comprising oxidant barrier material under anaerobic conditions. Preferably, the anaerobic conditions comprise atmospheric concentration of oxidant molecules less than about 1000 ppb, 100 ppb, or most preferably, less than about 10 ppb, and/or wherein the anaerobic conditions comprise a gas atmosphere selected from the group consisting of: argon, nitrogen, sulfur hexafluoride and carbon dioxide. Inert gases may be selected from any of the well-known gases used to exclude oxygen and/or carry out chemical reactions in a strict anaerobic environment. Inert gases useful with the present invention include but are not limited to: nitrogen, sulfur hexafluoride, carbon dioxide, and the noble gases (e.g., argon, xenon, etc.), or any combination of these inert gases.

Increased Storage Lifetime with Oxidant Barrier Packaging, Dehydration, and Oxidant Scavengers

Further, the above described oxidative protection methods of the present invention can be carried our wherein an oxidant barrier material is used for packaging. Oxidant barrier material can be selected from the list consisting of: glass, mylar, high-density polypropylene, aluminum foil, polyvinylidenchloride, polyester, polyamide and cellulose films, and all combination thereof.

Additionally, the methods of the present invention for increasing storage lifetime can be carried out wherein an oxidant scavenging material is included in the container from the group consisting of: a dessicant, an oxidant scavenging catalyst, or any combination thereof. Dessicants and oxidant scavenging catalysts useful with the present invention are well-known in the art. In one embodiment, dessicants and oxidant scavenging catalysts useful with the present invention may be selected from the group consisting of: sulfite and bisulfite, finely divided metals, heated metal elements, tannins, carbohydrazides, enzymes including glucose oxidase, and unsaturated organic compounds.

Oxidant barrier packaging materials useful with the present invention include any material capable of excluding the common oxidizing gases, e.g., oxygen, and ozone. Such materials include the packaging films well-known in the food science and pharmaceutical arts. Additionally, container systems used to maintain chemical and biochemical reagents under anaerobic conditions are well-known in the art. Oxidant barrier packaging materials include but are not limited to: glass, mylar, high-density polypropylene, aluminum foil, polyvinylidenchloride, polyester, polyamide and cellulose films, and all combination thereof.

In order that the invention may be more readily understood, reference is made to the following examples, which are intended to be illustrative of the invention, but are not intended to be limiting in scope.

### EXAMPLES

### Example 1

### Preparation of Ion Channel Sensor

This example illustrates the preparation of an ion channel sensor (ICS). The ICS includes a first and second lipid layer in a bilayer assembly tethered to a solid support. The ICS bilayer assembly includes gramicidin channels linked to antibodies that extend above the second layer surface. The ICS performance degrades over time, due in part to oxidation, and thus, provides a useful measure of methods for improving oxidative resistance of lipid layers.

1) Preparation of ICS Sealed Bilayer Assembly

Sensors were assembled using the following techniques. Gold coated polycarbonate slides (AMBRI, N.S.W., Australia) were soaked in absolute ethanol (200 proof) for 24 hours, followed by soaking in an ethanolic solution of first layer lipids for another 24 hours at room temperature. The slides were then rinsed twice with ethanol and bolted into 16-well stainless steel Hamilton blocks fitted with polypropylene well inserts. Ethanol (15 uL) was added immediately to each well to prevent drying, after which the Hamilton blocks were transferred to a Biomek 2000 liquid handler (Beckman Coulter, Fullerton, Calif.) where 15 μL of sealed (no gramicidin) second layer lipid solution, AM435 (3 mM DPEPC/GDPE (7:3) in ethanol, AMBRI, N.S.W., Australia) were added, followed by incubation for 3 minutes. At this point phosphate-buffered saline (PBS, 150 uL), pH 7.4, was added to each well. After an incubation period (40 seconds) the PBS was removed and followed by 3 additional rinse steps with PBS.

The parameters of the freshly assembled sealed lipid bilayers are measured using impedance spectroscopy scanning from 1000 to 0.1 Hz. A positive DC bias of 50 mV was applied and the voltage amplitude was 300 mV. The custom software program, Z32 (AMBRI, N.S.W., Australia) was used to compile impedance spectroscopy data. Typical values for the frequency of minimum phase (FminP) were 2-5 Hz. The Hamilton blocks were then returned to the Biomek where they are either converted to full sensors through the addition of the biochemistry layer (i.e., the gramicidin channels and antibody), or prepared for drying through removal of the PBS solution.

2) Addition of Ion Channels and Antibody to Lipid Bilayer

Sealed bilayer assemblies prepared as described above were converted to conducting biosensors through the addition of a biotinylated gramicidin derivative, gA5XB, which is commercially available from AMBRI (AMBRI, N.S.W., Australia). After temperature equilibration and vigorous mixing, a 5 μL aliquot of the gA5XB stock solution (6.31 μM in ethanol) was diluted into 15.8 mL of 1×PBS solution. Automated addition of 100 μL of the diluted gA5XB solution to Hamilton blocks was carried out using a Biomek liquid handler. Typical FminP values of 40-60 Hz were observed following this step.

The antibody components were then linked to the bilayer as follows: 100 μL of 84 nM streptavidin was added followed by 100 μL of 1 μM of newly prepared biotinylated antibody fragments (anti-hCG Fab's 103 and 106) with extensive PBS washing in between.

3) Vacuum Drying Process

Biosensors were dried following removal of most of the PBS solution in the Hamilton block wells. Either one or two stage drying was used. In the one stage drying process, membranes were dried under vacuum at 120 mTorr for 40 minutes at a constant temperature of 22° C.

The two stage drying process consists of the one stage process (120 mTorr for 40 minutes at 22° C.) followed by a second stage of 1-10 mTorr vacuum for 60 minutes with the temperature ramped up to 50° C. over the first 30 minutes and then ramped back down to 22° C. over the next 30 minutes. The two-stage vacuum drying process was used with membranes prepared for coating with trehalose barrier compound solution. The second stage of the drying process was performed at an elevated temperature under a higher vacuum. These conditions induce the trehalose to form a higher T_{g} glass and help immobilize the whole bilayer membrane.

In either drying process the biosensors are preferably kept under an inert atmosphere such as argon gas during all transfer steps.

4) Anaerobic Storage of Dried Biosensors

Anaerobic storage was carried out in an anaerobic chamber (Bactron model IV) in which the oxygen is actively scavenged by palladium catalyst and hydrogen. The atmosphere inside the chamber has the characteristics shown in Table 2:

TABLE 2
Anaerobic chamber characteristics

| Parameter | Level |
|---|---|
| Oxygen Level | 0 ppm |
| Relative Humidity | 30-40% |
| Nitrogen | 95% |
| Hydrogen | 2-5% |

Additionally, biosensors were stored in the anaerobic chamber sealed in polyethylene zip-lock bags containing silica desiccant which exposes the ICS membranes to an overall relative humidity of 0-14% during storage.

5) Biosensor Rehydration

Rehydration was carried out by addition of 100 μL PBS solution to all wells in the Hamilton block, followed by incubation at room temperature for 5 min, and five further buffer exchanges of the wells. Control biosensors (i.e., those not stored) were rehydrated immediately after drying (Day 0). Stored biosensors were rehydrated following storage under either anaerobic or ambient conditions for the designated time period.

6) Performance Measurement by hCG Gating

Blocks were incubated at 33° C. by placing them on a plate heater for 30 min. hCG dilutions to the wells were made according to Table 3 and were also equilibrated to 33° C. before analyte addition. Impedance spectroscopy measurements were started and 5 min of background drift data were collected. Gating response was measured for ˜15 min; measurements made with frequency range 1-1000 Hz.

TABLE 3
Dilution series of hCG used in biosensor gating experiments

| Well | hCG¹ (mIU/mL) |
|---|---|
| 8, 9 | 0 |
| 7, 10 | 30 |
| 6, 11 | 60 |
| 5, 12 | 90 |
| 4, 13 | 120 |
| 3, 14 | 150 |
| 2, 15 | 180 |
| 1, 16 | 0 |

¹hCG dilutions were pre-incubated in a 96-well plate at 33° C. for˜5min. and no more than 10 min.

7) Data Analysis

Raw data was analyzed using the custom software program Zg32.v1.19 (AMBRI, N.S.W., Australia). The software fits the gating response to an exponential plus drift algorithm. Large data sets were analyzed by using CONAN, an automated fitting program (AMBRI, N.S.W., Australia) to determine tau (τ), the exponential decay time constant. Fitted data was exported to an Excel spreadsheet and the data plotted using the 1/tau parameter versus hCG analyte concentration.

Performance of the sensors is determined by calculating the statistics for a standard curve generated by ICS biosensor signals generated with series of analyte dilutions. Performance relates to the value of the four criteria “slope,” “R²,” “X-error” and “% Duds” and the ability of the sensor to differentiate between different analyte concentrations. Stability is defined in terms of the four performance criteria. If at a given storage time 100% of the sensor blocks exceed the minimum values for slope and R² and 100% of the sensors do not exceed the maximum values of X-error and % Duds given below, then the sensor is considered stable. Reproducibility is defined as the percentage of performance parameters that were met by sets of biosensors. In most cases a set is defined as four Hamilton blocks.

A straight line that best fits the standard curve gives a description of the performance of the ICS biosensor array. The “slope” values are used to infer the responsiveness of the sensor design. “R²”, the coefficient of determination, describes the correlation in the sample between estimated signal and the actual signal. The R² is used to infer the linearity of the sensor design. “X-error,” the standard error for the x estimate, describes the range of potential analyte concentrations predicted for the measured ICS biosensor signal. It is used to estimate the detection limits. “% Dud” describes the rejection rate of the sensor array during the manufacturing. % Dud is determined by rejecting sensors based on statistical description of exponential fits to the signal decay curves. Automatic fitting filters are set in the data analysis package to reject sensors with goodness of fit below 0.998 and decay signal amplitude ratio more than 100%.

The performance of sensors was assessed with respect to the four key metrics according to the performance ranges listed in Table 4. Performance parameters were derived based on the average of four Hamilton blocks of biosensors, a unit termed a set. The number of measured parameters for a set falling within the specification range was recorded and used to determine sensor reproducibility. All parameters were given equal weight and thus a set that meets 2 of 4 key parameters was assigned a reproducibility of 50%.

TABLE 4
Specifications used to assess sensor performance and reproducibility¹.

| | In Spec | Out of Spec, but still functional | Out of Spec |
|---|---|---|---|
| Slope | ≧1.1 E−5 | 1.0E−5 < value < 1.1E−5 | ≦1.0 E−5 |
| R² | ≧0.8 | 0.7 < value < 0.8 | ≦0.7 |
| X-error | ≦40 | 40 < value < 50 | ≧50 |
| % Duds | ≦20 | 20 < value < 40 | ≧40 |

### Example 2

### Comparison of Anaerobic and Non-Anaerobic Storage of Ion-Channel Biosensors

This example illustrates the determination of comparative storage stability of lipid layer based ion-channel biosensors stored under anaerobic and non-anaerobic conditions.

Materials and Methods

Sealed ion channel biosensors were assembled in Hamilton blocks using standard Biomek robotic assembly techniques under ambient atmospheric conditions as described above in Example 1. Sensors were dried at 22° C. for 40 minutes at 120 mTorr vacuum using a Vertis Advantage Freeze Drier.

For anaerobic storage, after drying, the bell jar was sealed and transferred to the air lock of a Bactron IV anaerobic chamber (Sheldon Manufacturing, OR, U.S.A.). The air lock was evacuated and filled with nitrogen gas several times before introducing the bell jar containing the sensors into the main chamber. The bell jar was opened inside the anaerobic chamber and the sensors were sealed in zip lock bags along with 3 silica dessicant pouches. The sensors sealed in zip lock bags with dessicant were stored inside the anaerobic chamber for a period of one or two months.

For non-anaerobic storage, after drying, the biosensors simply were sealed in Mylar bags with a silica gel dessicant for a period of one or two months.

After the required storage time, the sensors were removed from the Mylar bags and rehydrated with PBS using the Biomek robot. Gramacidin insertion and biochemistry assembly (addition of streptavidin and hCG antibody FABs) were then performed on the Biomek robot to complete the assembly of the full biosensor.

The assembled biosensors were then challenged with 5 different concentrations of Human chorionic gonadotropin (hCG) hormone analyte and the impedance across the bilayer was monitored as a function of time to generate an impedance plot or gating curve. Gating response curves were then fit to a single exponential curve and a tau value was calculated for each analyte concentration. The parameter 1/tau was plotted as a function of hCG concentration and the slope. The parameters R², X-error and dud rate also were calculated in order to assess the performance of the biosensor.

Results

Table 5 lists the measured gating analyte response parameters for sensors stored for one month under non-anaerobic conditions followed by rehydration and gating with hCG analyte. The parameter ranges used to define a functional biosensor have been described above in Table 4. The results in Table 5 show that after one month, the 4 sets of ion channel sensor blocks met or exceeded only 38% (6 of 16) of the stability metrics used to define sensor performance.

TABLE 5
Performance of dried sensors stored under
non-anaerobic conditions for 1 month.

| 1 Month | Set 1¹ | Set 2 | Set 3 | Set 4 | Stability % |
|---|---|---|---|---|---|
| slope | 9.78E−07 | 8.74E−06 | 9.95E−06 | 9.60E−06 | 0 |
| R² | 0.030 | 0.856 | 0.622 | 0.633 | 25 |
| X-err | 70 | 27 | 44 | 43 | 25 |
| % duds | 8 | 14 | 11 | 11 | 100 |
| | 1/4 | 3/4 | 1/4 | 1/4 | 6/16 (38%) |

¹Each set comprises 4 blocks

As shown in Tables 6 and 7, the gating analyte response parameters for four sets of sensors stored under anaerobic conditions for one and two months respectively. It can be seen that at one month, the 4 sets of biosensors met or exceeded 88% (14 of 16) of the performance parameters, while 63% (5 of 8) of those performance parameters were met or exceeded by the 4 sets of biosensors stored for 2 months. Thus, storage under anaerobic conditions (nitrogen atmosphere in a mylar bag with dessicant) results in dramatically improved sensor performance.

TABLE 6
Performance of dried sensors stored under anaerobic conditions
for 1 month, followed by rehydration and gating with hCG analyte.

| 1 Month | set 1 | set 2 | set 3 | set 4 | Stability % |
|---|---|---|---|---|---|
| Slope | 2.45E−05 | 9.96E−06 | 1.80E−05 | 1.13E−05 | 75 |
| R² | 0.892 | 0.881 | 0.934 | 0.950 | 100 |
| Std Err X | 23 | 24 | 18 | 16 | 100 |
| % Duds | 8 | 3 | 11 | 22 | 75 |
| | 4/4 | 3/4 | 4/4 | 3/4 | 14/16 (88%) |

TABLE 7
Performance of dried sensors stored under anaerobic conditions
for 2 months, followed by rehydration and gating with hCG analyte.

| 2 Months | Anaerobic | Anaerobic 23° C. | Stability % |
|---|---|---|---|
| Slope | 1.89E−05 | 1.63E−05 | 100 |
| R² | 0.866 | 0.694 | 50 |
| Std Err X | 26 | 39 | 100 |
| % Duds | 36 | 22 | 0 |
| | 3/4 | 2/4 | 5/8 (63%) |

### Example 3

### Preparation and Performance of Biosensors with an Antioxidant Containing Lipid Layer

This example illustrates preparation of biosensors with an antioxidant containing lipid layer and improved performance characteristics of these biosensors following drying, storage and rehydration.

Materials and Methods

A) Preparation of Sealed Second Layer Antioxidant Cocktail

A mixture of the lipid soluble antioxidants, vitamin E (88 mg, 204 μmol) and butylated hydroxytoluene (BHT) (8.9 mg, 40.5 μmol), was dissolved in absolute ethanol (5 mL) resulting in a solution with final concentrations of 40.8 mM Vitamin E and 8.1 mM BHT. The solution was stored at 4° C. in the dark. A 5 μL aliquot of this solution was added to 2000 μL of AM435 sealed second layer lipid solution (AMBRI, Pty., Ltd., N.S.W., Australia) such that the final concentrations of vitamin E and BHT were 102 μM and 20.2 μM, respectively. The antioxidant solution was also used at double this antioxidant concentration (10 μL in 2 mL sealed second layer) without any negative impact on the performance of the resulting lipid bilayer biosensors.

A PBS solution containing 5% w/v of the water soluble antioxidant, ascorbic acid was prepared and adjusted to pH 7.4 with sodium hydroxide (6M). This water soluble antioxidant containing buffer was used in the assembly of the membrane biosensors together with the sealed second layer antioxidant cocktail.

B) Assembly of Antioxidant-Containing Sealed Biosensors.

Assembly of antioxidant-containing sealed biosensors was carried out according to the standard method for sealed bilayer biosensor assembly described in Example 1, except that the initial addition of buffer to the ethanolic second layer solution was performed using the ascorbic acid containing PBS solution. Subsequent washes were performed with standard PBS solution.

Following assembly, the performance parameters of the sealed biosensors were analyzed by impedance spectroscopy. The recorded parameters were similar to sealed biosensors without added antioxidants.

The freshly assembled antioxidant-containing biosensors were then subjected to the steps of volume reduction, vacuum drying and non-anaerobic storage in sealed Mylar bags in the presence of a silica gel dessicant as described in Example 2.

C) Measurement of Biosensor Performance Following Storage and Rehydration.

A set of eight sealed biosensors was assembled using antioxidant-containing second layer (5 μL in 2 mL) and were dried and stored under non-anaerobic conditions as described in Example 2. Rehydration of four sealed biosensors was performed immediately following drying (Day 0 time point) using the standard Biomek protocol (see general methods section), followed by the conversion of the sealed biosensors to full conducting biosensors through the addition of the biochemistry layer (Streptavidin then biotinylated anti-hCG antibodies).

The rehydrated, full biosensors were gated with hCG at 33° C. according to the standard protocol and the response measured using impedance spectroscopy. This procedure was repeated on day 6. Data was analyzed and compiled as described previously.

Three additional sets of eight biosensors were assembled, stored, rehydrated and gated at various time points ranging from 7 to 33 days.

Results

The standard biosensor metrics (Slope, R², X-error and % Duds) were determined and used to assess performance. As shown in Table 8, the measured performance metrics did not correlate with storage time, or exhibit the expected decrease in performance due to oxidative damage during storage. These results indicate that the lipid layers prepared with the lipid soluble antioxidant cocktail and water-soluble antioxidant buffer exhibited increased protection from oxidative damage.

TABLE 8
Improved performance of biosensors containing anti-oxidants.

| | Storage | | | | | |
|---|---|---|---|---|---|---|
| Exp | (days) | Slope | R² | X-error | % Dud | Stability % |
| 1 | 0 | 7.9E−06 | 0.97 | 12 | 20 | 75 (3/4) |
| 1 | 6 | 5.9E−06 | 0.78 | 33 | 44 | 25 (1/4) |
| 2 | 14 | 1.1E−05 | 0.93 | 18 | 3 | 100 (4/4) |
| 2 | 33 | 9.0E−06 | 0.94 | 17 | 8 | 75 (3/4) |
| 3 | 7 | 1.1E−05 | 0.96 | 15 | 16 | 100 (4/4) |
| 4 | 13 | 9.4E−06 | 0.91 | 21 | 17 | 75 (3/4) |
| 4 | 28 | 9.2E−06 | 0.93 | 19 | 28 | 50 (2/4) |

### Example 4

### Improved Resistance to Ozone Oxidation of Biosensors with an Antioxidant Containing Lipid Layer

Two sets of eight sealed biosensors were assembled with and without the antioxidant cocktail according to the methods described in Examples 1 and 3. The antioxidant-containing biosensors were made from second layer solution containing 10 μL of the antioxidant cocktail per 2 mL. The blocks were assembled to sealed membrane stage and dried.

Sensors were exposed to ozone in the 50-100 ppb range for 15 minutes. Ozone was generated using an Air-Zone XT-120 household ozone generator (Air-Zone Inc., Suffolk, Va.) housed in a Plexiglas box (30 cm×45 cm×50 cm) containing a small fan for circulation. Ozone levels were measured using a handheld Model 1108 Ozone meter (Ozone Solutions, Inc., Sioux Center, Iowa, USA) placed near to the sensors during exposure. The ozone generator was used on the lowest setting in pulse mode (40 seconds of O₃ generation per minute).

Following ozone exposure the sealed biosensors were rehydrated and the raw membrane parameters acquired. Rehydrated, sealed biosensors were converted to full biosensors through addition of biochemistry and gated with hCG.

Results

Data analysis was performed as described in Example 3 and results are listed in Table 9. The anti-oxidant containing biosensors exhibited 50% reproducibility (2/4 metrics within specification) whereas the control (without antioxidant) exhibited only 25% reproducibility. These results support the ability of antioxidant inclusion molecules to protect lipid layers from oxidative damage even when the lipid layer is dehydrated and exposed to the atmospheric ozone.

TABLE 9
Impact of ozone exposure (15 minutes at 0.05 PPM) on gating
performance of sensors with and without added antioxidant.

| | Control | Anti-Oxidant |
|---|---|---|
| Slope | 1.36E−06 | 7.02E−06 |
| R\hat{ }2 | 0.207 | 0.859 |
| Std Err X | 63 | 27 |
| % Duds | 0 | 33 |
| Stability % | 25 (1/4) | 50 (2/4) |

### Example 5

### Increased Resistance to Ozone Oxidation of Lipid-layer Biosensors Using Alternative Antioxidants, Protective Agents, and Barrier Compounds (DMSO, and Trehalose)

Materials and Methods

A set of eight dried, sealed ICS biosensors were assembled using various oxidative protection strategies. The conditions used for assembly consisted of two blocks of control biosensors (i.e., no added antioxidant) and two blocks with the added antioxidant cocktail as described in Example 3. In addition, two blocks were assembled using as an alternative antioxidant a first layer solution containing 5% dimethylsulfoxide (DMSO). Two blocks were assembled with no added antioxidant (as in Example 1) but following assembly (and before dehydration) were coated with a 10% aqueous solution of the barrier compound trehalose. The coating procedure involves adding a 10-15 μL aliquot of the 10% trehalose solution to each of the 16 wells of a Hamilton block, followed by drying to leave a glassy coating. This was followed by the standard two stage drying procedure resulting in a glassy coating on each well. Following dehydration, and without any storage period, each set of biosensors was exposed to ozone gas as described in Example 4. Rehydration and analysis by impedance spectroscopy was carried out as described in Example 1.

Results

The results shown in Table 10 demonstrate that antioxidant-containing lipid biosensors were considerably more resistant to ozone degradation than were control biosensors. Additionally, sensors coated with the barrier compound trehalose after assembly, or prepared with a lipid layer containing the antioxidant DMSO, were also less prone to oxidative damage relative to the control.

TABLE 10
Performance of sensors with various oxidative protection
following a 15 minute exposure to 0.05 ppm ozone.

| | | | Coating | |
|---|---|---|---|---|
| | | Antioxidant | with 10% | Coating with |
| | Control | Cocktail | Trehalose | 5% DMSO - 1 Hr |
| Slope | 1.36E−06 | 8.80E−06 | 1.01E−05 | 1.09E−05 |
| R\hat{ }2 | 0.207 | 0.938 | 0.844 | 0.677 |
| Std Err X | 63 | 18 | 28 | 40 |
| Duds | 0 | 3 | 1 | 6 |
| Wells | 32 | 32 | 32 | 32 |
| % Duds | 0 | 9 | 3 | 19 |
| Stability % | 25 (1/4) | 75 (3/4) | 100 (4/4) | 50 (2/4) |

### Example 6

### Storage Stability of Sealed ICS Biosensors Containing the Antioxidant Cocktail

A set of four blocks was assembled using the standard protocol for sealed ICS biosensors described in Example 1. Of the four, two were kept as control blocks and two blocks were prepared with antioxidant cocktail as described in Example 3.

Raw biosensor performance parameters were measured using impedance spectroscopy on the freshly assembled wet sensors and initial FminP values were observed to be between 3 and 5 Hz. Two blocks (one control and one with antioxidant) were dried per the standard protocol whereas the remaining two blocks were left in the wet state.

All four blocks were then stored for 3 days under ambient atmospheric conditions (i.e., 1 atm air, no inert gases). Following the storage period, the buffer remaining in the wet sensors was exchanged with fresh PBS (200 μL). The dry blocks were rehydrated by the addition of PBS (200 μL) with mixing. Raw performance parameters of all four blocks were then measured by impedance spectroscopy as described in Example 1.

Results

The effect of 3 days of air exposure on the FminP parameter for wet sensors with and without added antioxidant is shown in Table 11. Each value in Table 11 corresponds to the average of 8 wells in the Hamilton block. The value of the FminP parameter tends to increase as lipid bilayer membranes degrade resulting in a decrease in the electrical resistance across the membrane.

TABLE 11
Impact of 3 days of air exposure on wet sensors.

| Condition | |
|---|---|
| 3 Days Air Exposure | FminP |

| (Wet) | Wells 1-8 | Wells 9-16 |
|---|---|---|
| No Antioxidant | 11.2 (+/−5.3) Hz | 11.0 (+/−2.5) Hz |
| With Antioxidant | 3.3 (+/−0.8) Hz | 3.6 (+/−1.2) Hz |

The effect of 3 days of air exposure on the FminP parameter for dry sensors with and without added antioxidant is shown in Table 12. Each value in Table 12 corresponds to the average of 8 wells in the Hamilton block. The sensors prepared with lipid layers containing antioxidant were observed to have FminP values similar to freshly prepared blocks. The blocks prepared without antioxidant containing lipid layers had significantly higher FminP values, indicative of greater current leakage across the membrane caused by oxidative damage to the lipid layer. Additionally, the dried sensors were significantly more susceptible to loss of performance due to air exposure than the sensors stored wet.

TABLE 12
Impact of 3 days of air exposure on dry sensors.

| Condition | FminP | |
|---|---|---|
| 3 Days Air Exposure (Dry) | Wells 1-8 | Wells 9-16 |
| No Antioxidant | >1000 | 25 >1000 |
| With Antioxidant | 56.8 (+/−42.2) Hz | 250.1 (+/−336.3) Hz |

### Example 7

### Improved Stability of Self-Assembled Monolayers Containing Antioxidants

This example illustrates methods for improved stability of lipid self-assembled monolayers (SAMs) by inclusion of antioxidants or use of barrier compound coatings as described in the Examples for lipid bilayer biosensors.

Preparation and Assessment of Control SAM

A SAM is formed through contacting a clean gold surface with a 0.1 mmol to 5.0 mmol solution of an alkanethiol or an co-functional alkanethiol in ethanol. The SAM forms over a 1 to 24 hour period. The SAM on the gold substrate is then washed with ethanol and dried under a stream of dry nitrogen.

Following exposure to ozone and/or other oxidants, the protected SAM is washed with deionized water and dried under a stream of dry nitrogen. The stability of these monolayers is assessed through impedance spectroscopy, contact angle measurement and atomic force microscopy (AFM).

Preparation of Antioxidant Containing Sam with Barrier Compound Coating

A SAM is formed as described in the control experiment above except that the alkanethiol solution also contains the antioxidant cocktail of BHT and vitamin E at a concentration of about 1% w/w or less relative to the alkanethiol.

Following incubation and drying, the SAM is coated with trehalose in accordance with the procedure described in Examples 1 and 5 so as to leave a uniform glassy film following vacuum drying. Following exposure to ozone and other oxidants the protected SAMs are washed with deionized water and analyzed as for the control SAMs above.

As with the lipid bilayer assemblies described in Example 1-6, the inclusion of antioxidants acts to decrease the oxidative degradation of SAMs following exposure to ozone and other oxidants relative SAMs without antioxidants and/or oxidant barriers.

1. A method for preparing a lipid layer on a solid support, said method comprising:
providing a solid support;
contacting said solid support with a solution, wherein said solution comprises a lipid layer forming compound and at least one lipid-soluble antioxidant compound at a concentration of about 1 μM to about 10 mM.

2. The method of claim 1, wherein the solid support comprises a lipid monolayer attached to the surface of the solid support.

3. The method of claim 1, wherein the lipid layer forming compound is an amphiphilic molecule selected from the group consisting of: phospholipids, glycolipids, thiolipids, bolaamphiphiles, phytanyl lipids, ether lipids, and any combination thereof.

4. The method of claim 1, wherein said lipid soluble antioxidant compound is selected from the group consisting of: Vitamin E, tocopherols, tocotrienols, phenols, BHA, BHT, thiols, sulfides, disulfides, sulfoxides, hydroquinones, ascorbyl palmitate, phenylenediamines, gallates, thiocarbamates, and any combination thereof.

5. The method of claim 1, wherein said solution comprises Vitamin E and BHT.

6. The method of claim 1, wherein said method further comprises contacting said support with a barrier compound solution after contacting with the lipid layer forming compound.

7. The method of claim 6, wherein said barrier compound is selected from the group consisting of: trehalose, sucrose, mannitol, polyvinylalcohol, cationic polymers, starch, polystyrene sulfonate, polyethylene glycol, and polyethylene oxide.

8. The method of claim 7, wherein said method further comprises dehydrating the lipid layer to a relative humidity level of less than about 20%.

9. The method of claim 1, wherein said method further comprises contacting said solid support with an aqueous buffer solution comprising at least one water-soluble antioxidant compound at a concentration of at least about 0.005% to about 10% w/v.

10. A lipid layer on a solid support with increased resistance to oxidation made according to the method of claim 1.

11. A lipid layer on a solid support with increased resistance to oxidation comprising a close-packed layer of an amphiphilic molecule, wherein the layer includes at least one lipid soluble antioxidant compound at a concentration of about 0.1 ppm to about 5000 ppm.

12. The lipid layer of claim 11, wherein the lipid layer is the top layer of a bilayer, wherein the lower layer of the bilayer is attached to the surface of the solid support.

13. The lipid layer of claim 12, wherein the bilayer comprises an ion channel.

14. The lipid layer of claim 11, wherein the amphiphilic molecule is selected from the group consisting of: phospholipids, glycolipids, thiolipids, bolaamphiphiles, phytanyl lipids and ether lipids.

15. The lipid layer of claim 11, wherein the lipid soluble antioxidant compound is selected from the group consisting of: Vitamin E, tocopherols, tocotrienols, phenols, BHA, BHT, thiols, sulfides, disulfides, sulfoxides, DTT, DMSO, hydroquinones, ascorbyl palmitate, phenylenediamines, and gallates.

16. The lipid layer of claim 15, wherein the lipid layer comprises the antioxidant compounds: vitamin E and BHT.

17. The lipid layer of claim 11, wherein said lipid layer further comprises a barrier compound solution in contact with the close-packed layer of amphiphilic molecules.

18. The lipid layer of claim 17, wherein said barrier compound is selected from the group consisting of: trehalose, polyvinylalcohol, cationic polymers, starch, polystyrene sulfonate, polyethylene glycol, and polyethylene oxide.

19. A method for preparing a lipid layer on a solid support for storage, said method comprising:
providing a solid support comprising a lipid layer;
contacting the lipid layer with a barrier compound solution, wherein the barrier compound is selected from the group consisting of: trehalose, polyvinylalcohol, cationic polymers, starch, polystyrene sulfonate, polyethylene glycol, and polyethylene oxide.

20. The method of claim 19, wherein the method further comprises dehydrating the lipid layer to a relative humidity level of less than about 20% after contacting the lipid layer with a barrier compound solution.

21. The method of claim 20, wherein the method for dehydrating comprises: placing the lipid layer in a chamber under an atmospheric pressure of less than about 200 mTorr for at least about 30 minutes.

22. The method of claim 21, wherein the method further comprises: reducing the atmospheric pressure to between about 0.1 and 20 mTorr at 40° C. for at least about 30 minutes followed by storage under inert gas at a relative humidity between about 0.1% and about 15%.

23. The method of claim 19, wherein the method further comprises sealing the lipid layer in a container comprising oxidant barrier material under anaerobic conditions.

24. The method of claim 23, wherein the oxidant barrier material is selected from the list consisting of: glass, mylar, high-density polypropylene, aluminum foil, polyvinylidenchloride, polyester, polyamide and cellulose films combination thereof.

25. The method of claim 23, wherein the anaerobic conditions comprise a gas atmosphere selected from the group consisting of: argon, nitrogen, sulfur hexafluoride and carbon dioxide.

26. An ion channel sensor comprising:
a solid support with a conducting surface;
a lipid bilayer with first and second layers each comprising closely packed amphiphilic molecules, wherein the first layer is attached to the conducting surface of the solid support and comprises a first ionophore, and wherein the second layer comprises a second ionophore, and at least one lipid soluble antioxidant compound at a concentration of about 0.1 ppm to about 5000 ppm;
a plurality of recognition molecules covalently attached to the second ionophores, wherein the recognition molecules are capable of binding to an analyte.

27. An ion channel sensor product comprising:
an ion channel sensor sealed in an oxidant barrier material container under anaerobic conditions, wherein the ion channel sensor comprises:
a solid support with a conducting surface;
a lipid bilayer with first and second layers each comprising closely packed amphiphilic molecules, wherein the first layer is attached to the conducting surface of the solid support and comprises a first ionophore, and wherein the second layer comprises a second ionophore, and at least one lipid soluble antioxidant compound at a concentration of about 0.1 ppm to about 5000 ppm;
a plurality of recognition molecules covalently attached to the second ionophores, wherein the recognition molecules are capable of binding to an analyte.

28. The ion channel sensor product of claim 27, wherein said product further comprises a barrier compound in contact with the second layer of the lipid bilayer.

29. The ion channel sensor product of claim 27, wherein the lipid bilayer is dehydrated

30. The ion channel sensor product of claim 27, wherein relative humidity of the lipid bilayer is less than about 20%.

31. The ion channel sensor product of claim 27, wherein the water activity level of the lipid bilayer is between about 0.01 and about 0.2.

32. The ion channel sensor product of claim 27, wherein the fluorescence recovery of the lipid layer following photobleaching of the lipid layer is between about 20% and about 80% of that for a fully hydrated lipid layer.

33. The ion channel sensor product of claim 27, wherein the diffusion coefficient of lipids in the lipid layer is between about 10% and about 50% of that for a fully hydrated layer.
